(19) United States
(12) Patent Application Publication
(10) Pub. No.: US 2002/0137187 A1
(43) Pub. Date: Sep. 26, 2002

(54) APPARATUS FOR CULTIVATING MICROORGANISMS

(76) Inventor: NIELS HENRIK NORSKER, STIRLING (GB)

Correspondence Address: DANIEL A MONACO, DRINKER BIDDLE & REATH LLP, ONE LOGAN SQAURE, 18TH AND CHERRY STREETS, PHILADELPHIA, PA, 19103, US

(21) Appl. No.: 09/254,807

(22) PCT Filed: Sep. 11, 1997
(86) PCT No.: PCT/GB97/02452

(30) Foreign Application Priority Data
Sep. 12, 1996 (GB) 9619058.2

(51) Int. Cl. C12N 1/00; A01N 1/00

(52) U.S. Cl. 435/243; 435/284.1; 435/289.1; 435/283.1

(57) ABSTRACT

A method and apparatus for illuminating bioreactors is disclosed, comprising light sources emitting light at a flux density above 300 μEm⁻²sec⁻¹ in order to inhibit the growth of algae etc on the walls of the culture vessel.

The invention relates to apparatus for cultivating microorganisms, and in particular algae.

Conventional microalgal cultivation for hatchery operations is usually done in plastic bags. The total production cycle is quite laborious. The long propagation procedure from pure stock cultures to large production bags introduces a significant risk that ciliates, other protozoans or microalgae may take over. Apparent evidence of such a situation is that the culture bleaches to “crash” soon after. Some species of ciliates may double several times a day—a growth rate which exceeds that of aquaculture algae, and accounts for the familiar experience that a bag may look fine one afternoon, and has crashed the next morning.

In some situations, production cultures are on the verge of such a crash. One may be able to harvest the culture just before the ciliates take over—but, if delayed, the crash is most likely to follow. Also suboptimum growth conditions for the algae make a successful harvest less likely.

With high cultivation volumes elevated temperature is very often a problem and limits the amount of light the culture may be given, especially in warm environments.

Closed semicontinuous microalgal cultures in the scale of 1-20 liters are on the other hand not difficult to maintain. Aseptically set up and regularly diluted with sterile growth medium, and sparged with air eventually containing a little CO₂, the culture may be continued for extended periods until attached growth on the walls of the flask makes change of flask necessary.

However, a disadvantage of this semicontinuous process is that it is labour intensive and wall growth limits its usefulness.

Often the so called flashing light principle is used to enhance photosynthetic yield in microbial cultures.

According to this principle, the photosynthetic energy conversion of a given amount of visible light in a culture of photosynthetic microorganisms such as micro algae, is increased when the light is administered in high intensity pulses, separated by longer intervals of darkness.

The principle yields photosynthetic energy conversion increments with light emitted in pulses of a duration as little as μ seconds to minutes; the results being most pronounced at the shorter pulses.

A number of design approaches to investigate the effects of this principle have been made, including the use of stroboscopic light in small experimental cultures and rotating culture vessels, such as a rotating drum (described in Oswald et al. 1965, Proc. Am.Soc.Civil Eng. U(Sanit.Eng.), 91(SA4), 23) where the culture is kept as a thin film on the walls of the rotating drum by the centrifugal force, or as in the so called Couette device, where the culture is rotated in a narrow gap between a central rotor and an outer static wall. Ichimura (1976, U.S. Pat. No. 3,986,297) describes a non-translucent photosynthetic reactor tank where light is administered through dry wells in the tank. A flushing effect is obtained by rotating a shutter with a slit around the light source. Although the flashing light effect in this system may result in increased photosynthetic conversion of the actually emitted light, a similar overall effect can not be expected as the flashing essentially is brought about by shading the light source.

Neither of these systems have any production scale applications as the energy input and construction costs to establish and maintain such systems are not justified by the productivity increases.

A system inducing stratified turbulence in outdoor open race way tanks by the introduction of wing-like bafflers in the culture is described by Laws et al. 1983 Biotechnol. Bioeng.25,2319-2335; this system, however, is still structurally complex and can not be implemented in an indoor culture system as used as production cultures, producing for example micro algae for bivalve hatcheries and nurseries, marine fish hatcheries, and industrial micro algae applications, such as human nutritional products, pharmaceutical products etc.

Known production technology for these applications comprise either 50-500 liter transparent hard cylinders or plastic bags, open cubic or rectangular tanks, all illuminated and stirred in non-strategic manners using randomly injected air bubbles.

As used herein, the term “light” refers to electromagnetic radiation having wavelengths in the ultraviolet, visible and/or infrared regions of the electromagnetic spectrum.

In accordance with the present invention, apparatus for cultivating microorganisms comprises a vessel for containing microorganisms and a light source, the light source emitting light having a flux density greater than 300 μEm⁻²sec⁻¹.

Preferably, the apparatus further comprises a movement mechanism to generate relative movement between the light source and the microorganisms.

Typically, the movement mechanism comprises a stirring device which stirs the microorganisms in the vessel to generate the relative movement.

Typically, the stirring device may comprise an impeller type stirrer or an air lift stirrer mounted within the vessel.

Preferably, a number of light sources are provided. The light source or sources are typically arranged around the periphery of the vessel and are preferably outside the vessel and the vessel is substantially transparent to the light.

Typically, the light has a flux density of greater than 500 μEm⁻²sec⁻¹, preferably greater than 600 μEm⁻²sec⁻¹, and most preferably greater than 1000 μEm⁻²sec⁻¹.

Preferably, the light sources are spaced around the periphery and are arranged such that not all the periphery of the vessel is directly exposed to light; typically, the width of the light striking the vessel and the said relative movement is generated such that exposure of particular microorganisms in the vessel to light is not more than 2 seconds at a time.

Preferably, the light sources are equi-spaced around the periphery of the vessel.

Preferably, where the vessel is cylindrical, and a number of light sources “n” are provided with length “l” and width “b”, and the length “l” extends substantially parallel to the central axis of the cylinder, then n times l is less than or equal to ⅓ of the circumference of the cylinder.

Typically, the relative movement is relative rotation between microorganisms in the vessel and the light source, such that the rotations per minute is greater than 60 b(d2π) where b is the width of the light source and d is the diameter of the vessel, and preferably n times b is less than πd/4 where n is the number of light sources.

An example of apparatus for cultivating microorganisms in accordance with the invention will now be described with reference to the accompanying drawings, in which:—

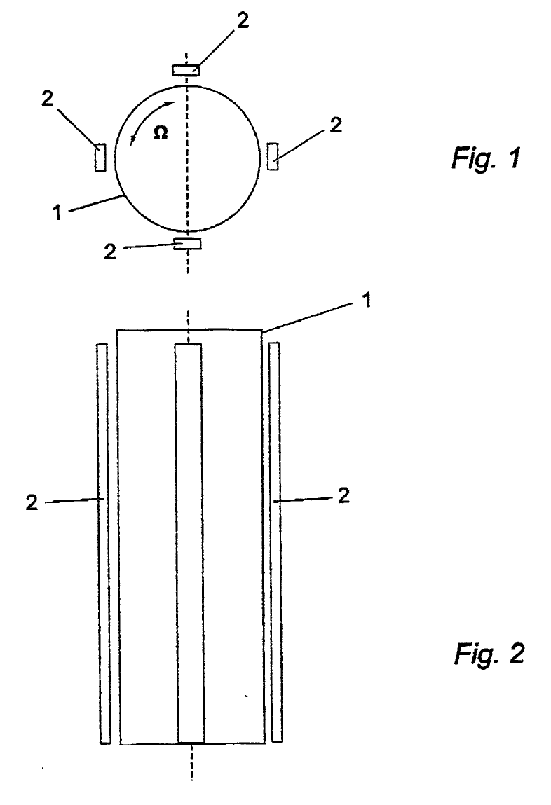

FIG. 1 is a schematic plan view of a bioreactor;

FIG. 2 is a schematic side view of the bioreactor shown in FIG. 1;

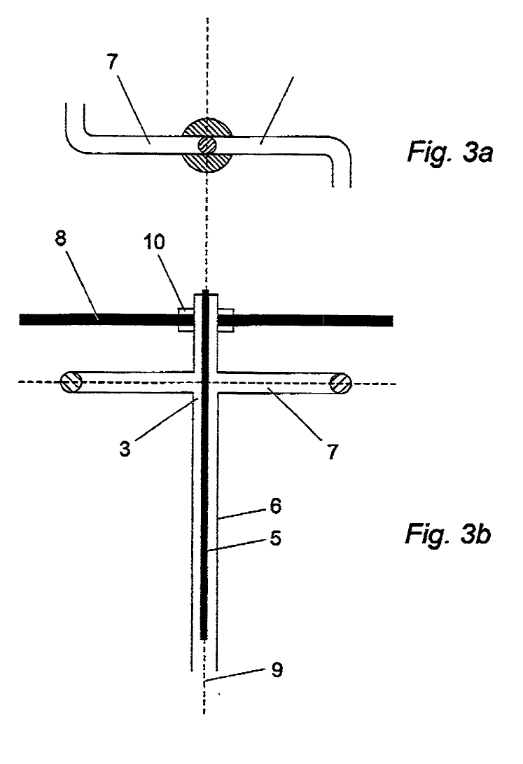

FIGS. 3a and 3b are a plan view and a side view respectively of an air lift stirrer for use with the bioreactor shown in FIGS. 1 and 2; and,

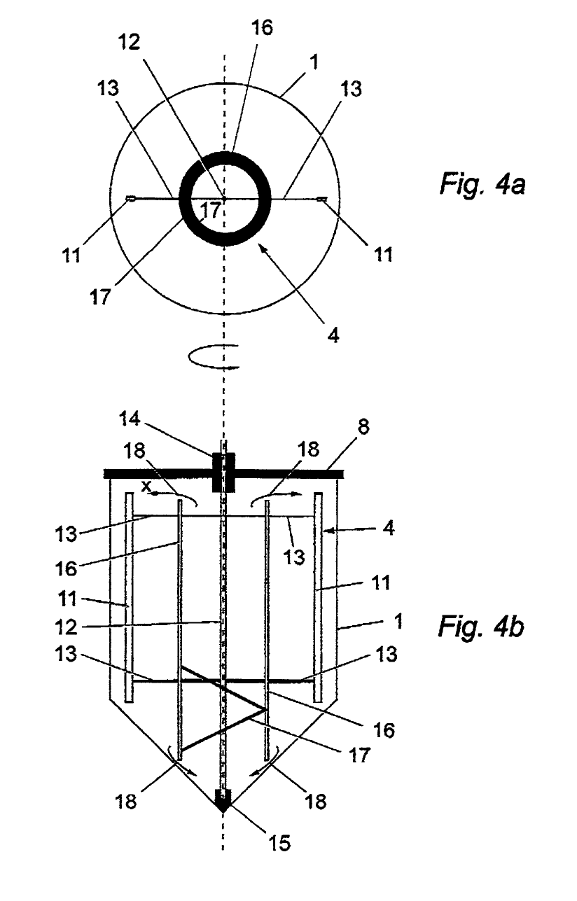

FIGS. 4a and 4b are a plan view and a side view respectively of an impeller type stirrer for use with the bioreactor shown in FIGS. 1 and 2.

FIG. 1 shows a bioreactor which comprises a cylindrical vessel 1 in which microorganisms in the form of an algae culture is contained (see FIGS. 1 and 2). Distributed around the outside of the vessel 1 are four light sources 2. Each light source 2 has a length l and a width b.

The algae within the vessel 1 are rotated by a stirrer mechanism at an angular speed of Ω revolutions per minute. The stirrer mechanism may be an air lift stirrer 3 (see FIGS. 3a and 3b) or an impeller type stirrer 4 (see FIGS. 4a and 4b).

Stirring of the culture contained in the cylindrical vessel 1 consists of two components;

1 rotation around the axis of the vessel 1; and,

2 a vertical displacement of the culture contained in the vessel 1.

The air lift stirrer 4 comprises a vertical tube 6 rotationally supported by a bearing 10 in a lid 8 of the vessel 1. Within the tube 6 is an air tube 5. In the air lift stirrer 3, as illustrated in FIGS. 3a and 3b, air bubbles are ejected from the air tube 5 which extends almost to the bottom of the vertical tube 6. The culture fluid is lifted up the tube 6 from the bottom of the tube 6 and ejected through the two horizontal arms 7 in a tangential direction The culture fluid in the vessel 1 is thereby rotated around a vertical axis 9 and is further more displaced in a vertical plug flow mode From top to bottom of the cylinder parallel to the axis 9.

In the impeller type stirrer 4, as illustrated in FIGS. 4a and 4b, vertically oriented rectangular impellers 11 are mounted on a central shaft 12 by supporting rods 13. The shaft 12 extends through the lid 8 for example by a magnetically coupled bearing 14. The shaft 12 is supported distally by a lower bearing 15. An inner rotating cylinder 16 is fixed to shaft 12 also by the impeller support rods 13. Part of the inner surface of the cylinder 16 is covered with a helical fringe 17. Hence, the inner cylinder 16 acts as a screw pump when it is rotated to move the culture fluid through the inner cylinder 16. In FIG. 4b the fluid direction in the inner cylinder 16, as indicated by arrows 18, is in the direction bottom to top. However, the direction may be reversed.

The flashing light principle is implemented in the bioreactor shown in FIGS. 1 and 2 by concentrating the light input in four vertical strands of light from the light sources 2, extending along preferably the entire culture-filled part of the cultivation cylinder.

The useful flux of visible light is 300 to 3000 μEm⁻²sec⁻¹; preferably higher than 600 μEm⁻²sec⁻¹ and most preferably higher than 1000 μEm⁻²sec⁻¹.

The volumetric irradiation (visible light) dose is 0.12E/liter/day<I/d(24 hrs) <0.36E/liter/day.

Above 1000 μEm⁻²sec⁻¹, an illuminated surface will be free of attached growth, from 600 μEm⁻²sec⁻¹ to 1000 μEm⁻²sec⁻¹, however, the attached growth problems are still greatly reduced.

Although four light sources 2 are shown, any number may be used. However, preferably two to four are used.

The incident light fields should be arranged as a number n of narrow strands each of length l and width b. The length l should cover a substantial part of the culture filled length of the vessel 1, preferably all of it. n×b should as small a fraction of the entire circumference of the cylinder as possible, and in all circumstances less than 25%.

The maximum circumference of the vessel 1 which should be covered by the light fields is given by the following equation:

n * b/dπ<0.25

The duration of each light pulse should be less than 2 seconds and to achieve this the angular speed Ω (rpm) of the culture should be greater than 60 b/(d2π).

By concentrating incident light in narrow patches with intensity above certain limits, wall growth of photosynthetic microorganisms in the reactor, which is a serious practical problem with previous types of photo bioreactors, is significantly reduced as the light intensity immediately below the wall of the culture cylinder is to high for attached biomass to be able to multiply and grow.

1 Apparatus for cultivating microorganisms comprising a vessel for containing microorganisms and a light source, the light source emitting light having a flux density greater than 300 μEm⁻²sec⁻¹.

2 Apparatus according to claim 1 having a movement mechanism to generate relative movement between the light source and the microorganisms.

3 Apparatus according to claim 2, wherein the movement mechanism comprises a stirring device which stirs the microorganisms in the vessel to generate the relative movement.

4 Apparatus according to claim 3, wherein the stirring device comprises an impeller-type stirrer or an air-lift stirrer mounted within the vessel.

5 Apparatus according to any preceding claim, wherein a number of light sources are provided.

6 Apparatus according to claim 5, wherein the light sources are spaced around the periphery of the vessel.

7 Apparatus according to any preceding claim, wherein the light source(s) is outside the vessel.

8 Apparatus according to any preceding claim, wherein the vessel is substantially transparent to the light.

9 Apparatus according to any preceding claim, wherein the light has a flux density of greater than 500 μEm⁻²sec⁻¹¹⁰.

10 Apparatus according to any preceding claim, wherein the light has a flux density of greater than 600 μEm⁻²sec⁻¹.

11 Apparatus according to any preceding claim, wherein the light has a flux density of greater than 1000 μEm⁻²sec⁻².

12 Apparatus according to any preceding claim, wherein several light sources are arranged such that not all the periphery of the vessel is directly exposed to light.

13 Apparatus according to any preceding claim, wherein the width of the light striking the vessel and the said relative movement is generated such that the period of exposure of particular microorganisms in the vessel to light is not more than 2 seconds.

14 Apparatus according to any preceding claim, wherein the vessel is cylindrical.

15 Apparatus according to claim 14, wherein a number of light sources “n” are provided with length “l” and width “b”, and their length “l” extends substantially parallel to the central axis of the cylinder, and wherein n×l is less than or equal to ⅓ of the circumference of the cylinder.

16 Apparatus according to any preceding claim, wherein the relative movement is relative rotation between microorganisms in the vessel and the light source.

17 Apparatus according to claim 16, wherein the rotations per minute is greater than 60 b(d2π) where b is the width of the light source and d is the diameter of the vessel.

18 Apparatus according to claim 17, wherein n×b is less than πd/4 where n is the number of light sources.

19 A method of illuminating a cylindrical bioreactor, the method comprising providing a number n of light sources of length l along the length of the cylinder, wherein n×l is less than or equal to ⅓ of the circumference of the cylinder, and wherein a unit volume of the contents adjacent the periphery of the cylinder is subjected to a regime of intermittent light and dark periods.
